(19) United States
(12) Patent Application Publication
(10) Pub. No.: US 2012/0330173 A1
(43) Pub. Date: Dec. 27, 2012

(54) APPARATUS AND METHOD FOR OBTAINING BIOMETRIC INFORMATION OF DRIVER

(75) Inventors: Woo Chul Park, Suwon-si (KR); Hyun Jong You, Hwaseong-si (KR); Jeong-Hwan Kim, Seoul (KR)

(73) Assignee: HYUNDAI MOTOR COMPANY, Seoul (KR)

(21) Appl. No.: 13/324,157

(22) Filed: Dec. 13, 2011

(30) Foreign Application Priority Data
Jun. 23, 2011 (KR) 10-2011-0061400

(51) Int. Cl. A61B 5/0456; A61B 5/00; A61B 5/0402

(52) U.S. Cl. 600/521; 600/509; 600/300

(57) ABSTRACT

An apparatus for obtaining biometric information of driver is provided. The apparatus includes a first biometric-signal-sensing unit that is installed in the steering wheel of an automobile and senses a driver's biometric signal therefrom. Additionally, a second biometric-signal-sensing unit is installed in the driver's seat of the automobile and also simultaneously senses a driver's biometric signal. As a means of control, a controller obtains biometric information selectively using biometric signals which satisfy a preset condition in obtaining biometric information based on biometric signals sensed by the first biometric-signal-sensing unit and biometric signals sensed by the second biometric-signal-sensing unit.

### CROSS-REFERENCES TO RELATED APPLICATIONS

This application claims priority to Korean patent application number 10-2011-0061400, filed on Jun. 23, 2011, which is incorporated by reference in its entirety herein.

### BACKGROUND OF THE INVENTION

1. Field of the Invention

The present invention relates to an apparatus and a method for obtaining biometric information of driver, and more particularly, to an apparatus and a method for obtaining biometric information of driver, in which biometric information obtained at a first position is substituted by biometric information obtained at a second position when an error is found in biometric information obtained at the first position. More specifically, the biometric information of driver is obtained through biosensors which are redundantly installed in a vehicle's steering wheel, driver's seat, safety belt of the driver's seat, etc.

2. Description of the Related Art

Recently, automobiles are not only being utilized as a means of transportation, but are also becoming a space where drivers can be provided various sets of information and services which are related to traffic, economy, culture and/or the driver's daily activities due to developments in the Internet and IT technologies.

As result, driver's safety and convenience has been greatly improved, and an individual's automobile has become an important asset for the driver's in respect to information, business and leisure in addition to transportation.

Automobile manufactures have also began applying ubiquitous-based medical services, that is, a u-healthcare systems, as well as technologies for driver's safety, in automobiles as well. In doing so, healthcare has been applied even while driving, thereby closing the gap between healthcare and our daily activities.

The u-healthcare system is a healthcare and medical service which is available to the driver regardless of time and location in the form of information technology and medical services combined, and remotely controls/diagnosis diseases and maintains and improves the health of the general public. Particularly, the u-healthcare system obtains biometric information from the driver while the driver is driving, analyzes health information of driver, sends feedback to driver or transmits the analyzed health information to the healthcare provider of driver.

Conventionally, a biometric-signal-sensing unit for sensing biometric signals of driver which are changed according to a given driving situation, a controller that determines driver's emotional state based on signals inputted from the biometric-signal-sensing unit, and outputs a control signal for adjusting driver's emotional state to the optimal state according to the result of the determination, and an emotion adjustment unit which controls driver's emotion by a control signal that is outputted from the controller, are provided.

In the conventional system, however, the biometric-signal-sensing unit is installed in a steering wheel. Hence, when driver removes his hand from the steering wheel, a biometric signal is not sensed, so the input signal at the time must be removed as noise, and thus it takes long time in obtain useful biometric information, and the accuracy of the obtained biometric information is reduced.

### SUMMARY OF THE INVENTION

The present invention has been made in view of the above problems, and provides an apparatus and a method for obtaining biometric information of driver, which can maintain continuity of biometric-signal sensing regardless of driver's movement or a faulty connection by substituting biometric information obtained at a first position with biometric information obtained at a second position. This information is obtained through biosensors which are redundantly installed in the steering wheel, driver's seat, and safety belt of the driver's seat, etc. of an automobile.

In accordance with an aspect of the present invention, an apparatus for obtaining biometric information of driver includes: a first biometric-signal-sensing unit (first unit) that is installed in a steering wheel of an automobile and is configured to sense a driver's biometric signal; a second biometric-signal-sensing unit that is installed in a driver's seat of the automobile and is configured to sense a driver's biometric signal; and a controller that is configured to obtain biometric information selectively using biometric signals which satisfy a preset condition in obtaining biometric information based on biometric signals sensed by the first biometric-signal-sensing unit and biometric signals sensed by the second biometric-signal-sensing unit.

In accordance with another aspect of the present invention, a method for obtaining driver's biometric information using a first biometric-signal-sensing unit that is installed in a steering wheel of an automobile and senses a driver's biometric signal, and a second biometric-signal-sensing unit that is installed at a driver's seat of the automobile and senses a driver's biometric signal includes: sensing a driver's biometric signal using a first biometric-signal-sensing unit; sensing a diver's biometric signal using a second biometric-signal-sensing unit; and obtaining biometric information selectively using biometric signals that satisfy a preset condition among biometric signals sensed by the first biometric-signal-sensing unit and biometric signals sensed by the second biometric-signal-sensing unit.

The above-described present invention can maintain continuity of biometric-signal even in a noisy section which is generated by driver movement or a faulty connection as biometric information obtained at a first position is substituted by biometric information obtained in a second position.

Particularly, according to the present invention, an electrocardiogram (ECG) sensor may be installed on a steering wheel and a driver's seat, respectively, so when driver detaches his hand from the steering wheel while obtaining ECG data through the ECG sensor installed at the steering wheel, the data may be substituted by ECG data obtained through the ECG sensor installed in the driver's seat, and the ECG data need not be re-obtained from the begin as would have been required in the conventional art.

### BRIEF DESCRIPTION OF THE DRAWINGS

The objects, features and advantages of the present invention will be more apparent from the following detailed description in conjunction with the accompanying drawings, in which:

FIG. 1 illustrates a configuration of an apparatus for obtaining biometric information of driver according to an exemplary embodiment of the present invention;

FIG. 2 illustrates a flowchart of a method for obtaining biometric information of driver according to an exemplary embodiment of the present invention;

FIG. 3 illustrates a result of performance analysis of an apparatus for obtaining biometric information of driver according to an exemplary embodiment of the present invention.

### DETAILED DESCRIPTION OF EXEMPLARY EMBODIMENTS

Exemplary embodiments of the present invention are described with reference to the accompanying drawings in detail. The same reference numbers are used throughout the drawings to refer to the same or like parts. Detailed descriptions of well-known functions and structures incorporated herein may be omitted to avoid obscuring the subject matter of the present invention.

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g., fuels derived from resources other than petroleum). As referred to herein, a hybrid vehicle is a vehicle that has two or more sources of power, for example both gasoline-powered and electric-powered vehicles.

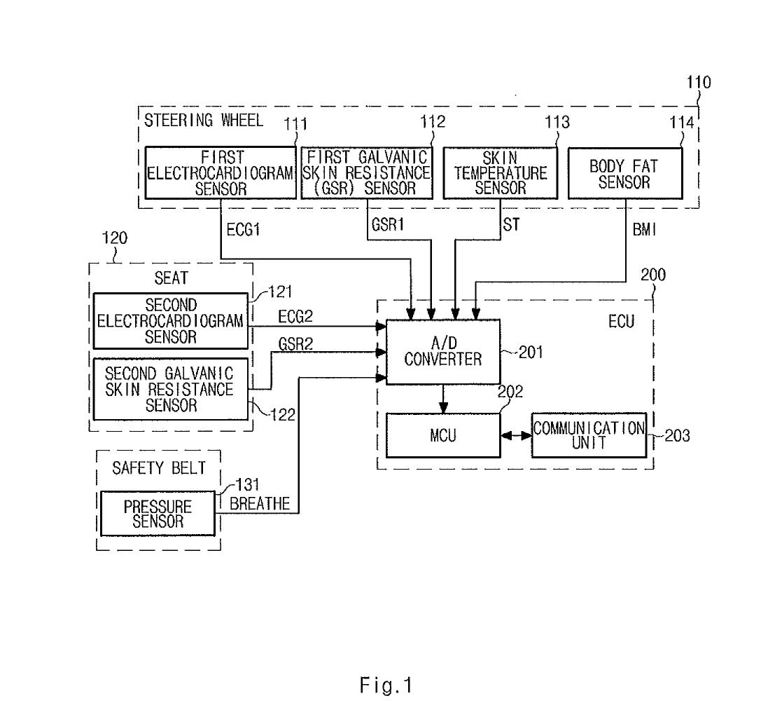

FIG. 1 illustrates a configuration of an apparatus for obtaining biometric information of driver according to an exemplary embodiment of the present invention. As illustrated in FIG. 1, an apparatus for obtaining biometric information of driver according to the present invention includes a first biometric-signal-sensing unit 110, a second biometric-signal-sensing unit 120 and an electronic control unit (ECU) 200.

Specifically, the first biometric-signal-sensing unit 110 is a sensor that is installed at or on the steering wheel of an automobile and senses a driver's biometric signal, and includes for example a first electrocardiogram (ECG) sensor 111, a first galvanic skin resistance (GSR) sensor 112, a skin temperature (ST) sensor 113 and a body fat sensor 114. Here, the first ECG sensor 111 includes an electrode on the right and left sides of the steering wheel, respectively, and obtains an electrocardiogram signal (ECG1) of driver from minute current that flows through the driver's body. This current is obtained through each electrode when a driver grabs the steering wheel with his both hands.

As an additional element, the first galvanic skin resistance (GSR) sensor 112 senses driver's galvanic skin resistance (GSR1), the skin temperature (ST) sensor 113 senses the skin temperature of the palms of driver, and the body fat sensor 114 measures the driver's body fat (BMI).

Next, the second biometric-signal-sensing unit 120 is installed at, in or on the driver's seat of an automobile. This unit 120 senses a biometric signal of driver, and includes a second electrocardiogram sensor 121 and a second galvanic skin resistance sensor 122. Here, the second electrocardiogram sensor 121 is an electrocardiogram-measuring sensor using a two-electrode method.

In particular, an electrode is attached on the right side and the left side of the back of a driver's seat which is in contact with the back of driver, respectively, and obtains an electrocardiogram signal (ECG2) from minute current which flows through the driver's body when the back of driver is in contact with the second electrocardiogram sensor 121. For example, in this exemplary embodiment, the electrode may be a textile electrode.

As additional elements, the second galvanic skin resistance sensor 122 senses driver's galvanic skin resistance, and a pressure sensor 131, which is installed at the driver's safety belt, may be configured to measure a driver's breathing rate.

Next, the electronic control unit (ECU) 200 includes an A/D converter 201, a micro control unit (MCU) 202 and a communication unit 203 in order to convert the driver's biometric signal into a digital value. The driver's biometric information is then obtained for biometric signals that satisfy preset conditions and are transmitted to a remote location in some embodiments. Here, the A/D converter 201 converts electrical biometric signals, which are obtained through the first biometric-signal-sensing unit 110, the second biometric-signal-sensing unit 120 and the pressure sensor 131, respectively, into digital values. The micro control unit 202 selects digital values that satisfy preset conditions from digital values which are converted in the A/D converter 201, respectively. The micro control unit 202 then obtains biometric information that is used in determining driver's emotional state and health state, etc. The communication unit 203 transmits biometric information obtained in the micro control unit 202 to a remote location, e.g., a server.

Particularly, the micro control unit 202 converts electrocardiogram signals (ECG1 and ECG2), which are transmitted from the first electrocardiogram sensor 111 installed at the steering wheel and the second electrocardiogram sensor 121 installed at the driver's seat, respectively, into digital values. Thereafter, when it is determined that the first electrocardiogram signal (ECG1) from the first electrocardiogram sensor 111 is noise, the micro control unit 202 substitutes the ECG1 with the second electrocardiogram signal (ECG2) from the second electrocardiogram sensor 121 in order to obtain biometric information.

Hence, the present invention maintains continuity of an electrocardiogram signal even when noise is generated due to a driver's failure to maintain contact with the electrodes of the first electrocardiogram sensor 111 which is installed at or on the steering wheel.

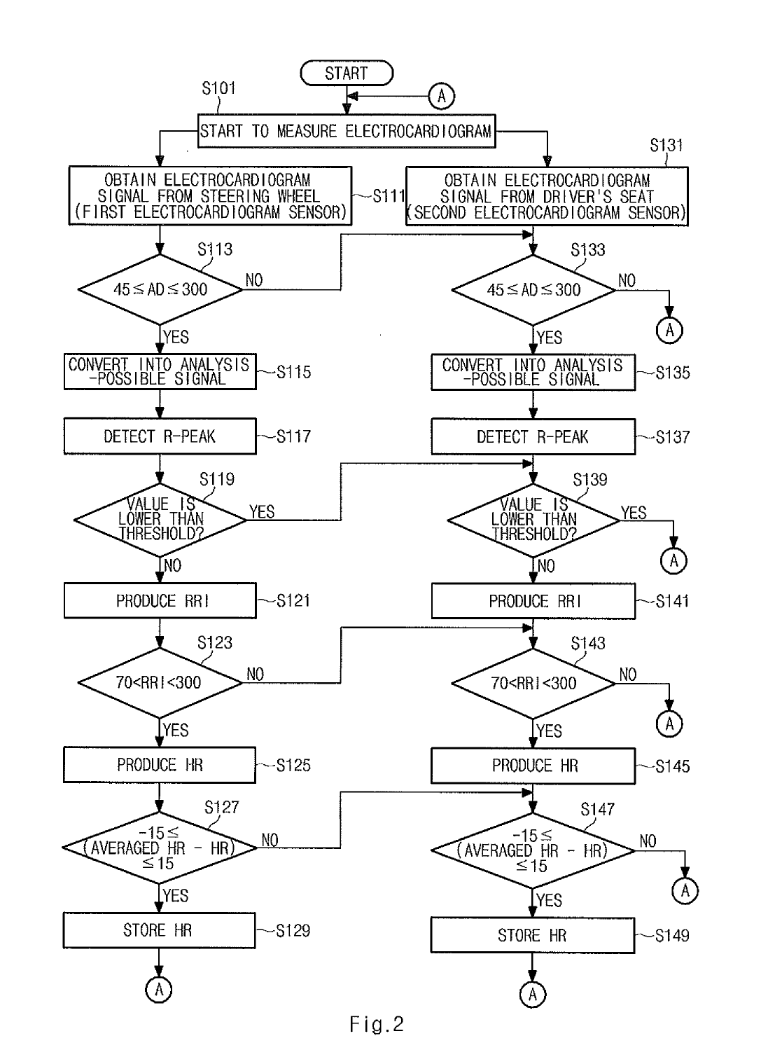

FIG. 2 illustrates a flowchart of a method for obtaining biometric information of driver according to an exemplary embodiment of the present invention. When a biometric information measurement mode, which is automatically or manually selected after driver takes a driver's seat, is executed, the first electrocardiogram sensor 111 installed at the steering wheel and the second electrocardiogram sensor 121 installed at the driver's seat are activated, thereby starting to measure the first electrocardiogram signal (ECG1) and the second electrocardiogram signal (ECG2), respectively (101).

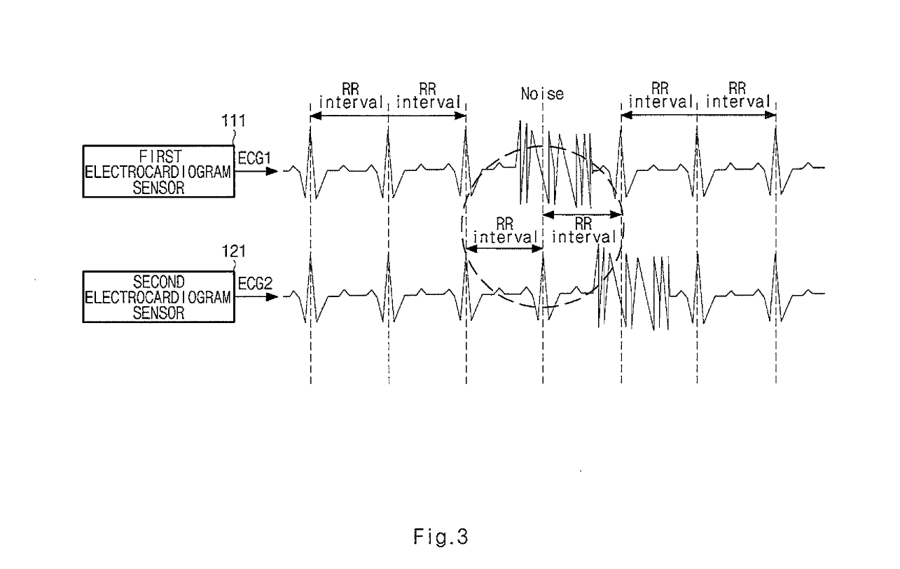

The electronic control unit 200 produces a digital value (AD), an R-peak value, an R-R interval (RRI) value and a heart rate (HR) value in consecutive order by selectively using the first electrocardiogram signal (ECG1) transmitted from the first electrocardiogram sensor 111 and the second electrocardiogram signal (ECG2) transmitted from the second electrocardiogram sensor 121 (111 to 127 and 131 to 147). Here, the RRI value refers to the number of sample points, which are included in one “R-R interval” in FIG. 3, that is, the number of points (sample points) of the portion expressed as a line within the “R-R interval”.

First, the A/D converter 201 receives the first electrocardiogram signal (ECG1) transmitted from the first electrocardiogram sensor 111 installed at the steering wheel, and converts the received first electrocardiogram signal into a digital value (AD) (111). When the converted digital values (AD) do not satisfy the preset measurement range (e.g., 45≦AD≦300) (113), it is determined that driver is failing to come in contact with the sensor. At this time, when the number of digital values (AD), where it is determined that driver failed to contact the sensor for a certain time period (e.g., 1 minute), is greater than 20, it is considered noise. In this instance, a compensation process (133-149) is executed.

A contact failure error of the first electrocardiogram sensor 111, which measures the electrocardiogram signal through each electrode of a steering wheel, occurs when both hands fail to contact each electrode installed on the right and left sides of the steering. In some cases only one of the driver's hands may fail to contact the electrode in order for there to be contact failure. Additionally, even though both hands accurately came in contacted the electrode at first, there may still be a contact failure when one hand fails to maintain contact with the electrode before obtaining all of the parameter values (R-peak value, RRI value and HR value). Here, the compensation process refers to a process that obtains digital values (AD) from the second electrocardiogram signal (ECG2) transmitted from the second electrocardiogram sensor 121 installed at the driver's seat, and produces a R-peak value, a RRI value and a HR value in consecutive order.

Further, when the converted digital values (AD) satisfy a preset measurement range (e.g., 45≦AD≦300) (113), the digital values are considered normal electrocardiogram signals and the noise having high frequency and low frequency elements is removed, and the first derivation and square root operation process is then executed and a R-peak is detected through, e.g., an envelope detection algorithm (115 to 117).

At this time, when the R-peak value detected through, e.g., the envelope detection algorithm, is lower than the threshold of the envelope (119), it is considered a contact failure error of the first electrocardiogram sensor 111 of driver. Accordingly, it is determined whether the R-peak value detected from the second electrocardiogram signal (ECG2) sensed at the second electrocardiogram sensor 121 is greater than the threshold of the envelope (139). When the R-peak value is greater than the threshold value, the process of producing an RRI value and an HR value through the second electrocardiogram signal (ECG2) is performed (141 to 145). At this time, when the R-peak value detected by the second electrocardiogram signal (ECG2) is also lower than the threshold of the envelope, the process returns to the initial electrocardiogram signal measurement step 101.

Further, when the R-peak value of the first electrocardiogram signal (ECG1) detected through the envelope detection algorithm is greater than the threshold of the envelope that is set, of the system begins to obtain the RRI value (121).

When the RRI of the obtained first electrocardiogram signal (ECG1) satisfies a preset condition (e.g., 70<RRI value<300) (123), the RRI value is considered normal, thereby producing the current heart rate (HR) (125). Here, the heart rate refers to the number of heartbeats over a 1 minute period of time.

When the RRI value of the first electrocardiogram signal (ECG1) does not satisfy a preset condition (e.g., 70<RRI value<300) (123), it is determined whether the RRI value obtained from the second electrocardiogram signal (ECG2) satisfies a preset condition (e.g., 70<RRI value<300) (143), and when the RRI value obtained from the second electrocardiogram signal (ECG2) satisfies the preset condition, the current heart rate (HR) is produced from the RRI value of the second electrocardiogram (ECG2), and the produced value is stored in e.g., a storage unit or on a remote server (145).

At this time, when the RRI value of the second electrocardiogram signal (ECG2) does not satisfy a preset condition, it is determined that the second electrocardiogram sensor 121 failed to obtain an electrocardiogram signal, and the process returns to the initial electrocardiogram signal measurement step 101 (denoted by “A”).

Thereafter, when the difference between the averaged heart rate (averaged HR) produced up to that instant in time and the averaged heart rate (HR) produced at step 125 (averaged HR−HR) is less than the criterion value (e.g., ±15), the heart rate is considered to be normal, and the heart rate is stored in either a memory, hard driver, remote server or the like (129) and the process returns to the initial electrocardiogram measurement step 101 (denoted by “A”).

At this time, when the difference between the averaged heart rate (averaged HR) produced up to that instant in time and the heart rate (HR) produced at step 125 (averaged HR−HR) is greater than the criterion value (e.g., ±15), the process moves to step 147. That is, when, e.g., 15≦(Averaged HR−HR)≦15 is not satisfied, the process moves to step 147.

At step 147, when the difference between the averaged heart rate (averaged HR) produced up to the time and the heart rate (HR) produced at step 145 (averaged HR−HR) is within the criterion value (e.g., ±15), the heart rate of the second electrocardiogram signal (ECG2) produced at step 145 is stored like the steps above, and the process then returns to the initial electrocardiogram measurement step 101.

At this time, when the difference between the averaged heart rate (averaged HR) produced up to the time and the heart rate (HR) produced at step 145 is greater than the criterion value (e.g., 115), the process promptly returns to the initial electrocardiogram measurement step 101.

A heart rate variability (HRV) is produced using each parameter (AD, R-peak, RRI value and HR value), which is biometric information produced by the above-described process, and the HRV is used in producing parameters such as standard deviation of all the normal RR intervals (SDNN), HRV-index and low frequency (LF)/high frequency (HF), etc., which are indexes for analysis, which can quantify whether the stress, emotion and autonomic nerve system is activated.

FIG. 3 illustrates the result of performance analysis of an apparatus for obtaining biometric information of driver according to the present invention, and is an electrocardiogram waveform diagram for explaining the process of determining and deciphering noise in a corresponding area of the first electrocardiogram signal (ECG1) when a contact failure error of the first electrocardiogram sensor occurs, and compensating with the electrocardiogram from the second electrocardiogram sensor. That is, the process of obtaining a digital converted value, a R-peak value, a RRI value and a HR value from the first electrocardiogram signal (ECG1) obtained in the first electrocardiogram sensor 111 is performed, and while obtaining the RRI value, the noise area is generated by failing to contact the first electrocardiogram sensor 111 of driver, thereby generating an error that consecutive RRI values cannot be obtained.

At this time, consecutive RRI values are produced by compensating with the RRI value obtained in the second electrocardiogram signal (ECG2) by the second electrocardiogram sensor 121 that is performing measurement simultaneously with the first electrocardiogram sensor 111.

In the present invention, obtaining biometric information can be arbitrarily set by user. That is, each parameter (AD, R-peak, RRI value and HR value) based on the second electrocardiogram signal (ECG2) obtained from the second electrocardiogram sensor 121, a setting range of a digital conversion value for determining whether there was a contact failure error in the first electrocardiogram signal and the second electrocardiogram signal, a threshold value range of a R-peak value, a normal detection range of a RRI value and a normal difference range between a HR and an averaged HR, etc. can be arbitrarily set by user.

Further, the detailed description of a process of obtaining a digital value (AD), a process of detecting an R-peak value, a process of producing a RRI value and a process of producing a HR value is omitted here because such processes are well-known to those skilled in the art.

Furthermore, the control logic of the present invention may be embodied as computer readable media on a computer readable medium containing executable program instructions executed by a processor, controller or the like. Examples of the computer readable mediums include, but are not limited to, ROM, RAM, compact disc (CD)-ROMs, magnetic tapes, floppy disks, flash drives, smart cards and optical data storage devices. The computer readable recording medium can also be distributed in network coupled computer systems so that the computer readable media is stored and executed in a distributed fashion, e.g., by a telematics server.

Although exemplary embodiments of the present invention have been described in detail hereinabove, it should be clearly understood that many variations and modifications of the basic inventive concepts herein taught which may appear to those skilled in the present art will still fall within the spirit and scope of the present invention, as defined in the appended claims.

What is claimed is:

1. An apparatus for obtaining biometric information of driver, the apparatus comprising:
a first biometric-signal-sensing unit installed in the steering wheel of an automobile and configured to sense a driver's biometric signal;
a second biometric-signal-sensing unit installed in the driver's seat of the automobile and configured to sense a driver's biometric signal; and
a controller configured to obtain biometric information selectively using biometric signals which satisfy a preset condition in obtaining biometric information based on biometric signals sensed by the first biometric-signal-sensing unit and biometric signals sensed by the second biometric-signal-sensing unit.

2. The apparatus of claim 1, wherein the first biometric-signal-sensing unit senses a driver's electrocardiogram signal (ECG1) through electrodes which are attached on right and left sides of the steering wheel, respectively.

3. The apparatus of claim 1, wherein the second biometric-signal-sensing unit senses a driver's electrocardiogram signal (ECG2) through textile electrodes which are attached on right and left sides of the driver's seat, respectively.

4. The apparatus of claim 1, wherein the controller comprises:
an analog/digital (A/D) converter configured to convert a first signal transmitted from the first biometric-signal-sensing unit into a first digital value, and converts a second signal transmitted from the second biometric-signal-sensing unit into a second digital value;
a micro control unit configured to select a digital value that satisfies a first condition among the first digital value and the second digital value converted by the A/D converter, and obtain biometric information based on the selected digital value; and
a communication unit configured to transmit biometric information obtained in the microcontroller to a server.

5. The apparatus of claim 4, wherein the controller detects a first R-peak value based on the first digital value when the first digital value satisfies the first condition, and detects a second R-peak value based on the second digital value when the digital value does not satisfy the first condition.

6. The apparatus of claim 5, wherein the controller returns to a process of determining whether the first condition is satisfied based on a biometric signal that is newly sensed through the first biometric-signal-sensing unit when the second digital value does not satisfy the first condition.

7. The apparatus of claim 5, wherein the controller detects a first R-R peak Interval (RRI) value based on the first R-peak value when the first R-peak value satisfies a second condition, and detects a second RRI value based on the second R-peak value when the first R-peak value does not satisfy the second condition.

8. The apparatus of claim 7, wherein the controller returns to a process of determining whether the first condition is satisfied based on a biometric signal that is newly sensed through the first biometric-signal-sensing unit when the second R-peak value does not satisfy the second condition.

9. The apparatus of claim 7, wherein the controller produces a first heart rate value based on the first RRI value when the first RRI value satisfies a third condition, and produces a second heart rate based on the second RRI value when the first RRI value does not satisfy the third condition.

10. The apparatus of claim 9, wherein the controller returns to a process of determining whether the first condition is satisfied based on a biometric signal that is newly sensed through the first biometric-signal-sensing unit when the second RRI value does not satisfy the third condition.

11. The apparatus of claim 9, wherein the controller stores the first heart rate on a remote server when the first heart rate satisfies a fourth condition, and produces a second heart rate when the first heart rate does not satisfy the fourth condition.

12. The apparatus of claim 11, wherein the controller returns to a process of determining whether the first condition is satisfied based on a biometric signal that is newly sensed through the first biometric-signal-sensing unit when the second heart rate does not satisfy the fourth condition.

13. A method for obtaining driver's biometric information using a first biometric-signal-sensing unit that is installed at the steering wheel of an automobile and senses a driver's biometric signal, and a second biometric-signal-sensing unit that is installed at the driver's seat of the automobile and senses a driver's biometric signal, the method comprising:
sensing a driver's biometric signal using a first biometric-signal-sensing unit;
sensing a diver's biometric signal using a second biometric-signal-sensing unit; and
obtaining, by a controller, biometric information selectively using biometric signals that satisfy a preset condition among biometric signals sensed by the first biometric-signal-sensing unit and biometric signals sensed by the second biometric-signal-sensing unit.

14. The method of claim 13, wherein obtaining the biometric information obtains a digital value (AD), a R-peak value, a RRI value and a heart rate in consecutive order selectively using biometric signals that satisfy a preset condition among biometric signals sensed by the first biometric-signal-sensing unit and biometric signals sensed by the second biometric-signal-sensing unit.

15. The method of claim 14, wherein obtaining the biometric information comprises:
converting, by analog to digital converter, an electrical biometric signal sensed by the first biometric-signal-sensing unit into a first digital value, and converting an electrical biometric signal sensed by the second biometric-signal-sensing unit into a second digital value;
detecting a first R-peak value based on the first digital value when the first digital value satisfies a first condition;
detecting a second R-peak value based on the second digital value when the first digital value does not satisfy the first condition; and
sensing the first biometric signal when the second digital value does not satisfy the first condition.

16. The method of claim 15, wherein obtaining the biometric information includes:
detecting a first R-R peak Interval (RRI) value based on the first R-peak value when the first R-peak value satisfies a second condition;
detecting a second RRI value based on the second R-peak value when the first R-peak value does not satisfy the second condition; and
sensing the first biometric signal when the second R-peak value does not satisfy the second condition.

17. The method of claim 16, wherein obtaining the biometric information includes:
producing a first heart rate based on the first RRI value when the first RRI value satisfies a third condition;
producing a second heart rate based on the second RRI value when the first RRI value does not satisfy the third condition; and
sensing the first biometric signal when the second RRI value does not satisfy the third condition.

18. The method of claim 17, wherein obtaining the biometric information includes:
storing, on a remote server, the first heart rate when the first heart rate satisfies a fourth condition;
storing, on the remote server, the second heart rate when the first heart rate does not satisfy the fourth condition; and
sensing the first biometric signal when the second heart rate does not satisfy the fourth condition.

19. A computer readable medium containing program instruction executed by a control unit, the computer readable medium comprising:
program instructions that obtain biometric information selectively using biometric signals that satisfy a preset condition among biometric signals sensed by a first biometric sensing unit and biometric signals sensed by a second biometric sensing unit installed in an automobile,
wherein when the first biometric sensor fails, the program instructions selectively substitute the signal from the first biometric sensing unit to the signal from the second biometric sensing unit.

20. The computer readable medium of claim 19, wherein obtaining the biometric information obtains a digital value (AD), a R-peak value, a RRI value and a heart rate in consecutive order selectively using biometric signals that satisfy a preset condition among biometric signals sensed by the first biometric sensing unit and biometric signals sensed by the second biometric sensing unit
